(19) United States
(12) Patent Application Publication
(10) Pub. No.: US 2011/0301433 A1
(43) Pub. Date: Dec. 8, 2011

(54) MENTAL STATE ANALYSIS USING WEB SERVICES

(76) Inventors: Richard Scott Sadowsky, Sturbridge, MA (US); Rana el Kaliouby, Newton, MA (US); Rosalind Wright Picard, Newtonville, MA (US); Oliver Orion Wilder-Smith, Holliston, MA (US); Panu James Turcot, Cambridge, MA (US); Zhihong Zheng, Zheng, MA (US)

(21) Appl. No.: 13/153,745

(22) Filed: Jun. 6, 2011

Related U.S. Application Data
(60) Provisional application No. 61/352,166, filed on Jun. 7, 2010.
(60) Provisional application No. 61/388,002, filed on Sep. 30, 2010.
(60) Provisional application No. 61/414,451, filed on Nov. 17, 2010.
(60) Provisional application No. 61/439,913, filed on Feb. 6, 2011.
(60) Provisional application No. 61/447,089, filed on Feb. 27, 2011.
(60) Provisional application No. 61/447,464, filed on Feb. 28, 2011.
(60) Provisional application No. 61/467,209, filed on Mar. 24, 2011.

(51) Int. Cl. A61B 5/00

(52) U.S. Cl. 600/300

(57) ABSTRACT

Analysis of mental states is provided using web services to enable data analysis. Data is captured for an individual where the data includes facial information and physiological information. Analysis is performed on a web service and the analysis is received. The mental states of other people may be correlated to the mental state for the individual. Other sources of information may be aggregated where the information may be used to analyze the mental state of the individual. Analysis of the mental state of the individual or group of individuals is rendered for display.

### RELATED APPLICATIONS

This application claims the benefit of U.S. provisional patent applications “Mental State Analysis Through Web Based Indexing” Ser. No. 61/352,166, filed Jun. 7, 2010, “Measuring Affective Data for Web-Enabled Applications” Ser. No. 61/388,002, filed Sep. 30, 2010, “Sharing Affect Data Across a Social Network” Ser. No. 61/414,451, filed Nov. 17, 2010, “Using Affect Within a Gaming Context” Ser. No. 61/439,913, filed Feb. 6, 2011, “Recommendation and Visualization of Affect Responses to Videos” Ser. No. 61/447,089, filed Feb. 27, 2011, “Video Ranking Based on Affect” Ser. No. 61/447,464, filed Feb. 28, 2011, and “Baseline Face Analysis” Ser. No. 61/467,209, filed Mar. 24, 2011. Each of the foregoing applications is hereby incorporated by reference in its entirety.

### FIELD OF INVENTION

This application relates generally to analysis of mental states and more particularly to evaluation of mental states using web services.

### BACKGROUND

The evaluation of mental states is key to understanding individuals but is also useful for therapeutic and business purposes. Mental states run a broad gamut from happiness to sadness, from contentedness to worry, and from excited to calm, as well as numerous others. These mental states are experienced in response to everyday events such as frustration during a traffic jam, boredom while standing in line, and impatience while waiting for a cup of coffee. Individuals may become rather perceptive and empathetic based on evaluating and understanding others' mental states but automated evaluation of mental states is far more challenging. An empathetic person may perceive another's being anxious or joyful and respond accordingly. The ability and means by which one person perceives another's emotional state may be quite difficult to summarize and has often been communicated as having a “gut feel.”

Many mental states, such as confusion, concentration, and worry, may be identified to aid in the understanding of an individual or group of people. People can collectively respond with fear or anxiety, such as after witnessing a catastrophe. Likewise, people can collectively respond with happy enthusiasm, such as when their sports team obtains a victory. Certain facial expressions and head gestures may be used to identify a mental state that a person is experiencing. Limited automation has been performed in the evaluation of mental states based on facial expressions. Certain physiological conditions may provide telling indications of a person's state of mind and have been used in a crude fashion as in an apparatus used for lie detector or polygraph tests.

There remains a need for improved evaluation of mental states in an automated fashion.

### SUMMARY

Analysis of mental states may be performed by evaluating facial expressions, head gestures, and physiological conditions exhibited by an individual. This analysis may aid in understanding consumer behavior, tailoring products more to user's desires, and improving websites and interfaces to computer programs. A computer implemented method for analyzing mental states is disclosed comprising: capturing data on an individual into a computer system wherein the data provides information for evaluating a mental state of the individual; receiving analysis from a web service wherein the analysis is based on the data on the individual which was captured; and rendering an output which describes the mental state of the individual based on the analysis which was received. The data on the individual may include one of a group comprising facial expressions, physiological information, and accelerometer readings. The facial expressions may further comprise head gestures. The physiological information may include one of a group comprising electrodermal activity, heart rate, heart rate variability, and respiration. The physiological information may be collected without contacting the individual. The mental state may be one of a cognitive state and an emotional state. The web service may comprise an interface which includes a server that is remote to the individual and cloud-based storage. The method may further comprise indexing the data on the individual through the web service. The indexing may include categorization based on valence and arousal information. The method may further comprise receiving analysis information on a plurality of other people wherein the analysis information allows evaluation of a collective mental state of the plurality of other people. The analysis information may include correlation for the mental state of the plurality of other people to the data which was captured on the mental state of the individual. The correlation may be based on metadata from the individual and metadata from the plurality of other people. The analysis which is received from the web service may be based on specific access rights. The method may further comprise sending a request to the web service for the analysis. The analysis may be generated just in time based on a request for the analysis. The method may further comprise sending a subset of the data which was captured on the individual to the web service. The rendering may be based on data which is received from the web service. The data which is received may include a serialized object in a form of JavaScript Object Notation (JSON). The method may further comprise deserializing the serialized object into a form for a JavaScript object. The rendering may further comprise recommending a course of action based on the mental state of the individual. The recommending may include one of a group comprising modifying a question queried to a focus group, changing an advertisement on a web page, editing a movie which was viewed to remove an objectionable section, changing direction of an electronic game, changing a medical consultation presentation, and editing a confusing section of an internet-based tutorial.

In some embodiments, a computer program product embodied in a computer readable medium for analyzing mental states may comprise: code for capturing data on an individual into a computer system wherein the data provides information for evaluating a mental state of the individual; code for receiving analysis from a web service wherein the analysis is based on the data on the individual which was captured; and code for rendering an output which describes the mental state of the individual based on the analysis which was received. In embodiments, a system for analyzing mental states may comprise: a memory which stores instructions; one or more processors attached to the memory wherein the one or more processors, when executing the instructions which are stored, are configured to: capture data on an individual wherein the data provides information for evaluating a mental state of the individual; receive analysis from a web service wherein the analysis is based on the data on the individual which was captured; and render an output which describes the mental state of the individual based on the analysis which was received.

Various features, aspects, and advantages of various embodiments will become more apparent from the following further description.

### BRIEF DESCRIPTION OF THE DRAWINGS

The following detailed description of certain embodiments may be understood by reference to the following figures wherein:

FIG. 1 is a diagram of a system for analyzing mental states.

FIG. 2 is a flowchart for obtaining and using data in mental state analysis.

FIG. 3 is a graphical rendering of electrodermal activity.

FIG. 4 is a graphical rendering of accelerometer data.

FIG. 5 is a graphical rendering of skin temperature data.

FIG. 6 shows an image collection system for facial analysis.

FIG. 7 is a flowchart for performing facial analysis.

FIG. 8 is a diagram describing physiological analysis.

FIG. 9 is a diagram describing heart rate analysis.

FIG. 10 is a flowchart for performing mental state analysis and rendering.

FIG. 11 is a flowchart describing analysis of the mental response of a group.

FIG. 12 is a flowchart for identifying data portions which match a selected mental state of interest.

FIG. 13 is a graphical rendering of mental state analysis along with an aggregated result from a group of people.

FIG. 14 is a graphical rendering of mental state analysis.

FIG. 15 is a graphical rendering of mental state analysis based on metadata.

### DETAILED DESCRIPTION

The present disclosure provides a description of various methods and systems for analyzing people's mental states. A mental state may be a cognitive state or an emotional state and these can be broadly covered using the term affect. Examples of emotional states include happiness or sadness. Examples of cognitive states include concentration or confusion. Observing, capturing, and analyzing these mental states can yield significant information about people's reactions to various stimuli. Some terms commonly used in evaluation of mental states are arousal and valence. Arousal is an indication on the amount of activation or excitement of a person. Valence is an indication on whether a person is positively or negatively disposed. Determination of affect may include analysis of arousal and valence. Affect may also include facial analysis for expressions such as smiles or brow furrowing. Analysis may be as simple as tracking when someone smiles or when someone frowns. Beyond this, recommendations for courses of action may be made based on tracking when someone smiles or demonstrates other affect.

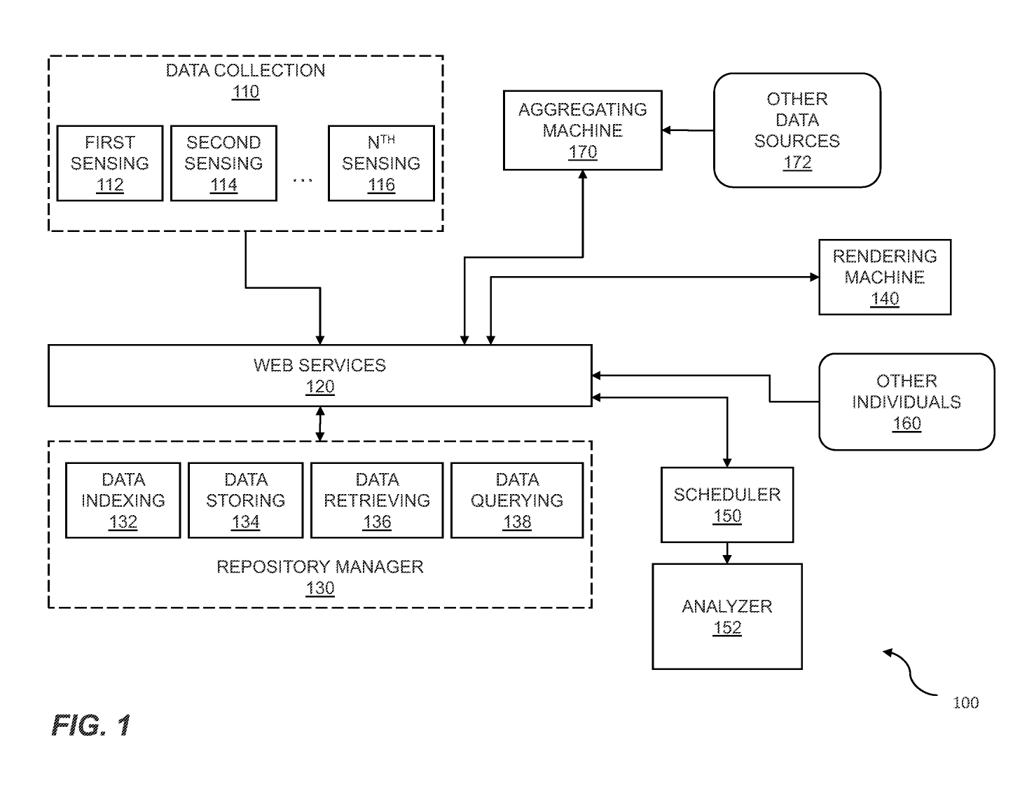

The present disclosure provides a description of various methods and systems associated with performing analysis of mental states. A mental state may be an emotional state or a cognitive state. Examples of emotional states may be happiness or sadness. Examples of cognitive states may be concentration or confusion. FIG. 1 is a diagram of a system 100 for analyzing mental states. The system may include data collection 110, web services 120, a repository manager 130, an analyzer 152, and a rendering machine 140. The data collection 110 may be accomplished by collecting data from a plurality of sensing structures such as a first sensing 112, a second sensing 114, through an n^{th} sensing 116. This plurality of sensing structures may be attached to an individual, be in close proximity to the individual, or may view the individual. These sensing structures may be adapted to perform facial analysis. The sensing structures may be adapted to perform physiological analysis which may include electrodermal activity or skin conductance, accelerometer, skin temperature, heart rate, heart rate variability, respiration, and other types of analysis of a human being. The data collected from these sensing structures may be analyzed in real time or may be collected for later analysis, based on the processing requirements of the needed analysis. The analysis may also be performed “just in time.” A just-in-time analysis may be performed on request, where the result is provided when a button is clicked on in a web page, for instance. Analysis may also be performed as data is collected so that a time line, with associated analysis, is presented in real time while the data is being collected or with little or no time lag from the collection. In this manner the analysis results may be presented while data is still being collected on the individual.

The web services 120 may comprise an interface which includes a server that is remote to the individual and cloud-based storage. Web services may include a web site, ftp site, or server which provides access to a larger group of analytical tools for mental states. The web services 120 may also be a conduit for data that was collected as it is routed to other parts of the system 100. The web services 120 may be a server or may be a distributed network of computers. The web services 120 may provide a means for a user to log in and request information and analysis. The information request may take the form of analyzing a mental state for an individual in light of various other sources of information or based on a group of people which correlate to the mental state for the individual of interest. In some embodiments, the web services 120 may provide for forwarding data which was collected to one or more processors for further analysis.

The web services 120 may forward the data which was collected to a repository manager 130. The repository manager may provide for data indexing 132, data storing 134, data retrieving 136, and data querying 138. The data which was collected through the data collection 110, through for example a first sensing 112, may be forwarded through the web services 120 to the repository manager 130. The repository manager can, in turn, store the data which was collected. The data may be indexed, through web services, with other data that has been collected on the individual on which the data collection 110 has occurred or may be indexed with other individuals whose data has been stored in the repository manager 130. The indexing may include categorization based on valence and arousal information. The indexing may include ordering based on time stamps or other metadata. The indexing may include correlating the data based on common mental states or based on a common experience of individuals. The common experience may be viewing or interacting with a web site, a movie, a movie trailer, an advertisement, a television show, a streamed video clip, a distance learning program, a video game, a computer game, a personal game machine, a cell phone, an automobile or other vehicle, a product, a web page, consuming a food, and so forth. Other experiences for which mental states may be evaluated include walking through a store, through a shopping mall, or encountering a display within a store.

Multiple ways of indexing may be performed. The data, such as facial expressions or physiological information may be indexed. One type of index may be a tightly bound index where a clear relationship exists which may be useful in future analysis. One example is time stamping of the data in hours, minutes, seconds, and perhaps in certain cases fractions of a second. Other examples include a project, client, or individual being associated with data. Another type of index may be a looser coupling where certain possibly useful associations may not be self-evident at the start of an effort. Some examples of these types of indexing may include employment history, gender, income, or other metadata. Another example may include the location where the data was captured, for instance in the individual's home, workplace, school, or other setting. Yet another example may include information on the person's action or behavior. Instances of this type information include whether a person performed a check out operation while on a website, whether they filled in certain forms, what queries or searches they performed, and the like. The time of day when the data was captured might prove useful for some types of indexing as might be the work shift time when the individual normally works. Any sort of information which might be indexed may be collected as metadata. Indices may be formed in an ad hoc manner and retained temporarily while certain analysis is performed. Alternatively, indices may be formed and stored with the data for future reference. Further, metadata may include self-report information from the individuals on which data is collected.

Data may be retrieved through accessing the web services 120 and requesting data which was collected for an individual. Data may also be retrieved for a collection of individuals, for a given time period, or for a given experience. Data may be queried to find matches for a specific experience, for a given mental response or mental state, or for an individual or group of individuals. Associations may be found through queries and various retrievals which may prove useful in a business or therapeutic environment. Queries may be made based on key word searches, based on time frame, or based on experience.

In some embodiments, a display is provided using a rendering machine 140. The rendering machine 140 may be part of a computer system which is part of another component of system 100, may be part of the web services 120, or may be part of a client computer system. The rendering may include graphical display of information collected in the data collection 110. The rendering may include display of video, electrodermal activity, accelerometer readings, skin temperature, heart rate, and heart rate variability. The rendering may also include display of mental states. In some embodiments, the rendering may include probabilities of certain mental states. The mental state for the individual may be inferred based on the data which was collected and may be based on facial analysis of activity units as well as facial expressions and head gestures. For instance, concentration may be identified by a furrowing of eye brows. An elevated heart rate may indicate being excited. Reduced skin conductance may correspond to arousal. These and other factors may be used to identify mental states which may be rendered in a graphical display.

The system 100 may include a scheduler 150. The scheduler 150 may obtain data that came from the data collection 110. The scheduler 150 may interact with an analyzer 152. The scheduler 150 may determine a schedule for analysis by the analyzer 152 where the analyzer 152 is limited by computer processing capabilities where the data cannot be analyzed in real time. In some embodiments aspects of the data collection 110, the web services 120, the repository manager 130, or other components of the system 100 may require computer processing capabilities for which the analyzer 152 may be used. The analyzer 152 may be a single processor or may be multiple processors or may be a networked group of processors. The analyzer 152 may include various other computer components such as memory and the like to assist in performing the needed calculations for the system 100. The analyzer 152 may communicate with the other components of the system 100 through the web services 120. In some embodiments, the analyzer 152 may communicate directly with the other components of the system. The analyzer 152 may provide an analysis result for the data which was collected from the individual wherein the analysis result is related to the mental state of the individual. In some embodiments, the analyzer 152 provides results on a just-in-time basis. The scheduler 150 may request just-in-time analysis by the analyzer 152.

Information from other individuals 160 may be provided to the system 100. The other individuals 160 may have a common experience with the individual on which the data collection 110 was performed. The process may include analyzing information from a plurality of other individuals 160 wherein the information allows evaluation of the mental state of each of the plurality of other individuals 160 and correlating the mental state of each of the plurality of other individuals 160 to the data which was captured and indexed on the mental state of the individual. Metadata may be collected on each of the other individuals 160 or on the data collected on the other individuals 160. Alternatively, the other individuals 160 may have a correlation for mental states with the mental state for the individual on which the data was collected. The analyzer 152 may further provide a second analysis based on a group of other individuals 160 wherein mental states for the other individuals 160 correlate to the mental state of the individual. In other embodiments, a group of other individuals 160 may be analyzed with the individual on whom data collection was performed to infer a mental state that is a response of the entire group and may be referred to as a collective mental state. This response may be used to evaluate the value of an advertisement, the likeability of a political candidate, how enjoyable a movie is, and so on. Analysis may be performed on the other individuals 160 so that collective mental states of the overall group may be summarized. The rendering may include displaying collective mental states from the plurality of individuals.

In one embodiment, a hundred people may view several movie trailers with facial and physiological data being captured from each. The facial and physiological data may be analyzed to infer the mental states of each individual and the collective response of the group as a whole. The movie trailer which has the greatest arousal and positive valence may be considered to motivate viewers of the movie trailer to be positively pre-disposed to go see the movie when it is released. Based on the collective response the best movie trailer may then be selected for use in advertizing an upcoming movie. In some embodiments, the demographics of the individuals may be used to determine which movie trailer is best suited for different viewers. For example, one movie trailer may be recommended where teenagers will be the primary audience. Another movie trailer may be recommended where the parents of the teenagers will be the primary audience. In some embodiments, webcams or other cameras can be used to analyze the gender and age of people as they interact with media. Further, IP addresses may be collected indicating geography where analysis is being collected. This information and other information can be included as metadata and used as part of the analysis. For instance, teens who are up past midnight on Friday nights in an urban setting might be identified as a group for analysis.

In another embodiment, a dozen people may opt in for having web cameras observe facial expressions and have physiological responses collected while they are interacting with a web site for a given retailer. The mental states of each of the dozen people may be inferred based on their arousal and valence analyzed from the facial expressions and physiological responses. Certain web page designs may be understood by the retailer to cause viewers to be more favorable to specific products and even to come more quickly to a buying decision. Alternatively, web pages which cause confusion may be replaced with web pages which may cause viewers to respond with confidence.

An aggregating machine 170 may be part of the system 100. Other sources of data 172 may be provided as input to the system 100 and may be used to aid in the mental state evaluation for the individual on whom the data collection 110 was performed. The other data sources 172 may include news feeds, Facebook™ pages, Twitter™, Flickr™, and other social networking and media. The aggregating machine 170 may analyze these other data sources 172 to aid in the evaluation of the mental state of the individual on which the data was collected.

In one example embodiment, an employee of a company may opt in to a self assessment program where his or her face and electrodermal activity are monitored while performing job duties. The employee may also opt in to a tool where the aggregator 170 reads blog posts, and social networking posts for mentions of the job, company, mood or health. Over time the employee is able to review social networking presence in context of perceived feelings for that day at work. The employee may also see how his or her mood and attitude may affect what is posted. One embodiment could be fairly non-invasive, such as just counting the number of social network posts, or as invasive as pumping the social networking content through an analysis engine that infers mental state from textual content.

In another embodiment, a company may want to understand how news stories about the company in the Wall Street Journal™ and other publications affects employee morale and job satisfaction. The aggregator 170 may be programmed to search for news stories mentioning the company and link them back to the employees participating in this experiment. A person doing additional analysis may view the news stories about the company to provide additional context to each participant's mental state.

In yet another embodiment, a facial analysis tool may process facial action units and gestures to infer mental states. As images are stored, metadata may be attached such as the name of the person whose face is in a video that is part of the facial analysis. This video and metadata may be passed through a facial recognition engine and be taught the face of the person. Once the face is recognizable to a facial recognition engine, the aggregator 170 may spider across the Internet, or just to specific web sites such as Flickr™ and Facebook™, to find links with the same face. The additional pictures of the person located by facial recognition may be resubmitted to the facial analysis tool for an analysis to provide deeper insight into the subject's mental state.

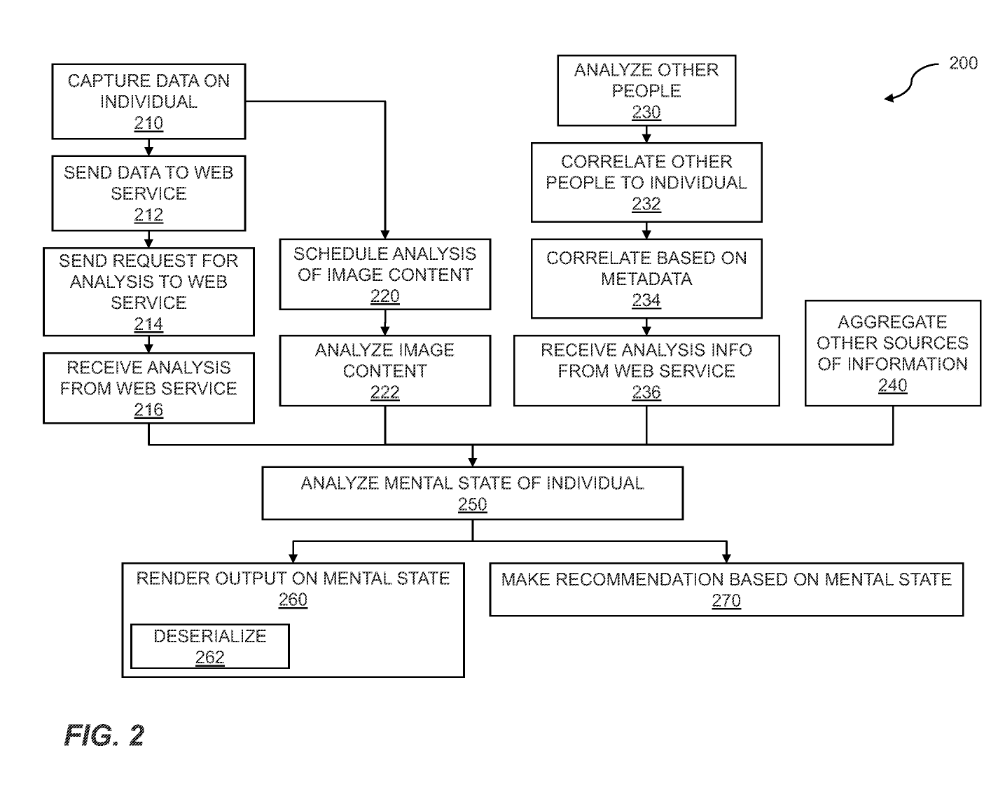

FIG. 2 is a flowchart for obtaining and using data in mental state analysis. The flow 200 describes a computer implemented method for analyzing mental states. The flow may begin by capturing data on an individual 210 into a computer system, wherein the data provides information for evaluating the mental state of the individual. The data which was captured may be correlated to an experience by the individual. The experience may be one of the group comprising interacting with a web site, a movie, a movie trailer, a product, a computer game, a video game, personal game console, a cell phone, a mobile device, an advertisement, or consuming a food. Interacting with may refer to simply viewing or may mean viewing and responding. The data on the individual may further include information on hand gestures and body language. The data on the individual may include facial expressions, physiological information, and accelerometer readings. The facial expressions may further comprise head gestures. The physiological information may include electrodermal activity, skin temperature, heart rate, heart rate variability, and respiration. The physiological information may be obtained without contacting the individual such as through analyzing facial video. The information may be captured and analyzed in real time, on a just-in-time basis, or on a scheduled analysis basis.

The flow 200 continues with sending the data which was captured to a web service 212. The data sent may include image, physiological, and accelerometer information. The data may be sent for further mental state analysis or for correlation with other people's data, or other analysis. In some embodiments, the data which is sent to the web service is a subset of the data which was captured on the individual. The web services may be a web site, ftp site, or server which provides access to a larger group of analytical tools and data relating to mental states. The web services may be a conduit for data that was collected on other people or from other sources of information. In some embodiments, the process may include indexing the data which was captured on a web service. The flow 200 may continue with sending a request for analysis to the web service 214. The analysis may include correlating the data which was captured with other people's data, analyzing the data which was captured for mental states, and the like. In some embodiments, the analysis is generated just in time based on a request for the analysis.

The flow 200 continues with receiving analysis from the web service 216 wherein the analysis is based on the data on the individual which was captured. The analysis received may correspond to that which was requested, may be based on the data captured, or may be some other logical analysis based on the mental state analysis or data captured recently.

In some embodiments, the data which was captured includes images of the individual. The images may be a sequence of images and may be captured by video camera, web camera still shots, thermal imager, CCD devices, phone camera, or other camera type apparatus. The flow 200 may include scheduling analysis of the image content 220. The analysis may be performed real time, on a just-in-time basis, or scheduled for later analysis. Some of the data which was captured may require further analysis beyond what is possible in real time. Other types of data may require further analysis as well and may involve scheduling analysis of a portion of the data which was captured and indexed and performing the analysis of the portion of the data which was scheduled. The flow 200 may continue with analysis of the image content 222. In some embodiments, analysis of video may include the data on facial expressions and head gestures. The facial expressions and head gestures may be recorded on video. The video may be analyzed for action units, gestures, and mental states. In some embodiments, the video analysis may be used to evaluate skin pore size which may be correlated to skin conductance or other physiological evaluation. In some embodiments, the video analysis may be used to evaluate pupil dilation.

The flow 200 may include analysis of other people 230. Information from a plurality of other individuals may be analyzed wherein the information allows evaluation of the mental state of each of the plurality of other individuals and correlating the mental state of each of the plurality of other individuals to the data which was captured and indexed on the mental state of the individual. Evaluation may also be allowed for a collective mental state of the plurality of other individuals. The other individuals may be grouped based on demographics, based on geographical locations, or based on other factors of interest in the evaluation of mental states. The analysis may include each type of data captured on the individual 210. Alternatively analysis on the other people 230 may include other data such as social media network information. The other people, and their associated data, may be correlated to the individual 232 on which the data was captured. The correlation may be based on common experience, common mental states, common demographics, or other factors. In some embodiments, the correlation is based on metadata 234 from the individual and metadata from the plurality of other people. The metadata may include time stamps, self reporting results, and other information. Self reporting results may include an indication of whether someone liked the experience they encountered, such as for example a video that was viewed. The flow 200 may continue with receiving analysis information from the web service 236 on a plurality of other people wherein the information allows evaluation of the mental state of each of the plurality of other people and correlation of the mental state of each of the plurality of other people to the data which was captured on the mental state of the individual. The analysis which is received from the web service may be based on specific access rights. A web service may have data on numerous groups of individuals. In some cases mental state analysis may only be authorized on one or more groups, for example.

The flow 200 may include aggregating other sources of information 240 in the mental state analysis effort. The sources of information may include news feeds, Facebook™ entries, Flickr™, Twitter™ tweets, and other social networking sites. The aggregating may involve collecting information from the various sites which the individual visits or for which the individual creates content. The other sources of information may be correlated to the individual to help determine the relationship between the individual's mental states and the other sources of information.

The flow 200 continues with analysis of the mental states of the individual 250. The data which was captured, the image content which was analyzed, the correlation to the other people, and other sources of information which were aggregated may each be used to infer one or more mental states for the individual. Further, a mental state analysis may be performed for a group of people including the individual and one or more people from the other people. The process may include automatically inferring a mental state based on the data on the individual that was captured. The mental state may be a cognitive state. The mental state may be an emotional state. A mental state may be a combination of cognitive and affective states. A mental state may be inferred or a mental state may be estimated along with a probability for the individual being in that mental state. The mental states that may be evaluated may include happiness, sadness, contentedness, worry, concentration, anxiety, confusion, delight, and confidence. In some embodiments, an indicator of mental state may be as simple as tracking and analyzing smiles.

Mental states may be inferred based on physiological data, accelerometer readings, or on facial images which are captured. The mental states may be analyzed based on arousal and valence. Arousal can range from being highly activated, such as when someone is agitated, to being entirely passive, such as when someone is bored. Valence can range from being very positive, such as when someone is happy, to being very negative, such as when someone is angry. Physiological data may include electrodermal activity (EDA) or skin conductance or galvanic skin response (GSR), accelerometer readings, skin temperature, heart rate, heart rate variability, and other types of analysis of a human being. It will be understood that both here and elsewhere in this document, physiological information can be obtained either by sensor or by facial observation. In some embodiments, the facial observations are obtained with a webcam. In some instances an elevated heart rate indicates a state of excitement. An increased level of skin conductance may correspond to being aroused. Small, frequent accelerometer movement readings may indicate fidgeting and boredom. Accelerometer readings may also be used to infer context such as, for example, working at a computer, riding a bicycle, or playing a guitar. Facial data may include facial actions and head gestures used to infer mental states. Further, the data may include information on hand gestures or body language and body movements such as visible fidgets. In some embodiments these movements may be captured by cameras or by sensor readings. Facial data may include tilting the head to the side, leaning forward, a smile, a frown, as well as many other gestures or expressions. Tilting of the head forward may indicate engagement with what is being shown on an electronic display. Having a furrowed brow may indicate concentration. A smile may indicate being positively disposed or being happy. Laughing may indicate enjoyment and that a subject has been found to be funny. A tilt of the head to the side and a furrow of the brows may indicate confusion. A shake of the head negatively may indicate displeasure. These and many other mental states may be indicated based on facial expressions and physiological data that is captured. In embodiments physiological data, accelerometer readings, and facial data may each be used as contributing factors in algorithms that infer various mental states. Additionally, higher complexity mental states may be inferred from multiple pieces of physiological data, facial expressions, and accelerometer readings. Further, mental states may be inferred based on physiological data, facial expressions, and accelerometer readings collected over a period of time.

The flow 200 continues with rendering an output which describes the mental state 260 of the individual based on the analysis which was received. The output may be a textual or numeric output indicating one or more mental states. The output may be a graph with a timeline of an experience and the mental states encountered during that experience. The output rendered may be a graphical representation of physiological, facial, or accelerometer data collected. Likewise, a result may be rendered which shows a mental state and the probability of the individual being in that mental state. The process may include annotating the data which was captured and rendering the annotations. The rendering may display the output on a computer screen. The rendering may include displaying arousal and valence. The rendering may store the output on a computer readable memory in the form of a file or data within a file. The rendering may be based on data which is received from the web service. Various types of data can be received including a serialized object in the form of JavaScript Object Notation (JSON) or in an XML or CSV type file. The flow 200 may include deserializing 262 the serialized object into a form for a JavaScript object. The JavaScript object can then be used to output text or graphical representations of the mental states.

In some embodiments, the flow 200 may include recommending a course of action based on the mental state 270 of the individual. The recommending may include modifying a question queried to a focus group, changing an advertisement on a web page, editing a movie which was viewed to remove an objectionable section, changing direction of an electronic game, changing a medical consultation presentation, editing a confusing section of an internet-based tutorial, or the like.

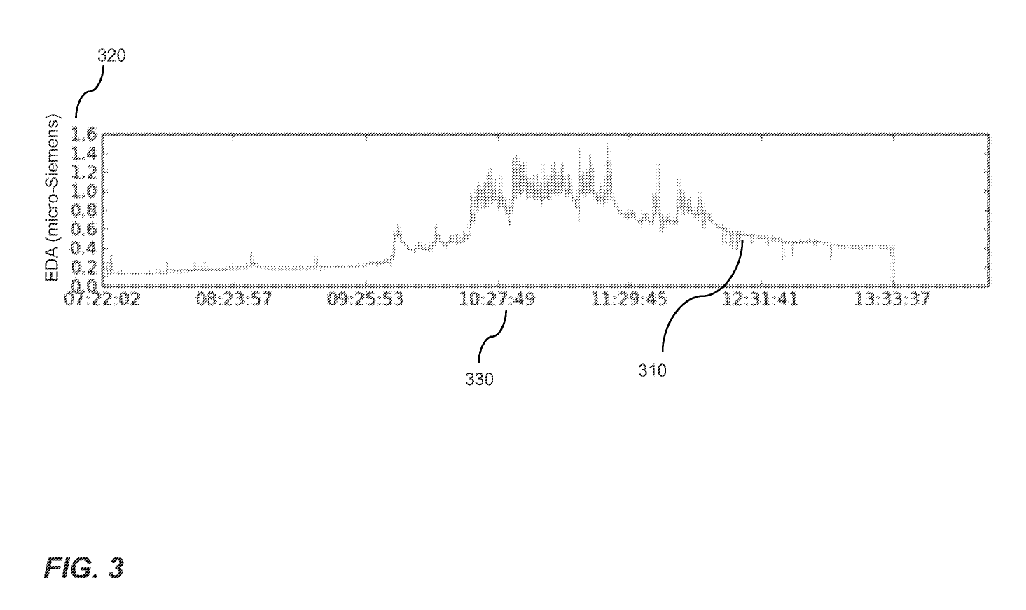

FIG. 3 is a graphical rendering of electrodermal activity. Electrodermal activity may include skin conductance which, in some embodiments, is measured in the units of micro-Siemens. A graph line 310 shows the electrodermal activity collected for an individual. The value for electrodermal activity is shown on the y-axis 320 for the graph. The electrodermal activity was collected over a period of time and the timescale 330 is shown on the x-axis of the graph. In some embodiments, electrodermal activity for multiple individuals may be displayed when desired or shown on an aggregated basis. Markers may be included and identify a section of the graph. The markers may be used to delineate a section of the graph that is or can be expanded. The expansion may cover a short period of time on which further analysis or review may be focused. This expanded portion may be rendered in another graph. Markers may also be included to identify sections corresponding to specific mental states. Each waveform or timeline may be annotated. A beginning annotation and an ending annotation may mark the beginning and end of a region or timeframe. A single annotation may mark a specific point in time. Each annotation may have associated text which was entered automatically or entered by a user. A text box may be displayed which includes the text.

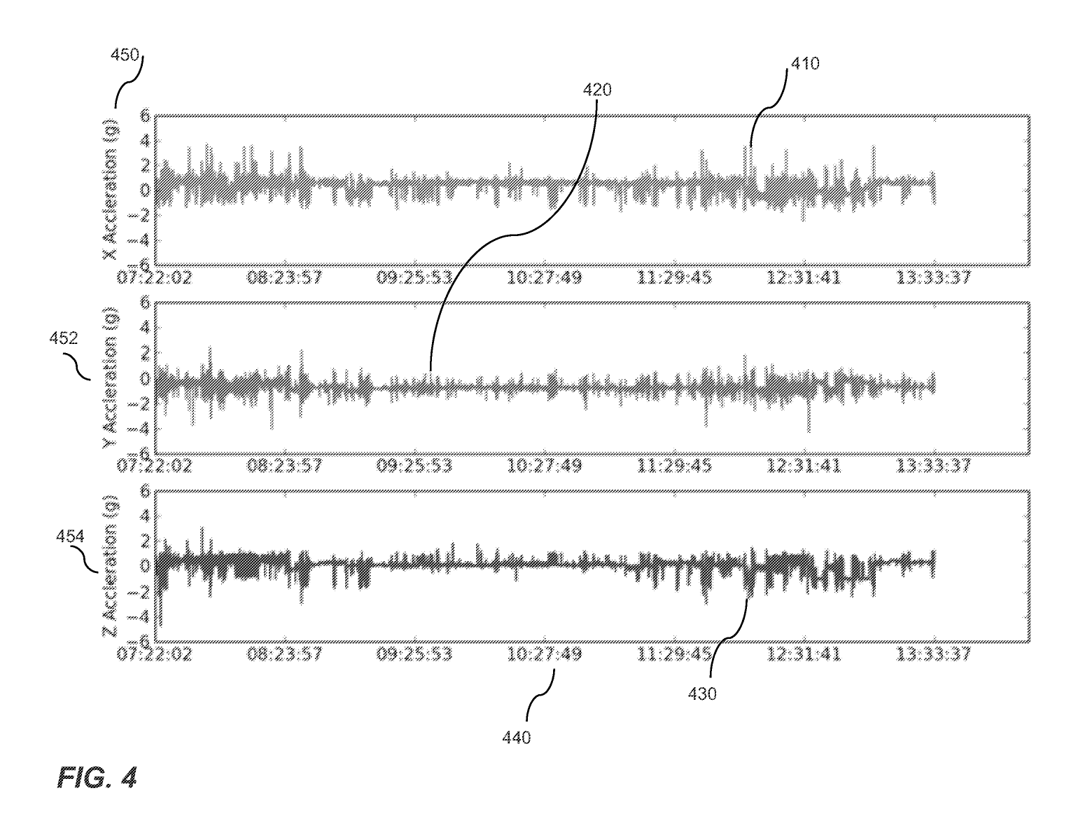

FIG. 4 is a graphical rendering of accelerometer data. One, two, or three dimensions of accelerometer data may be collected. In the example of FIG. 4, a graph for x-axis accelerometer readings are shown in a first graph 410, a graph for y-axis accelerometer readings are shown in a second graph 420, and a graph for z-axis accelerometer readings are shown in a third graph 430. The timestamps for the corresponding accelerometer readings are shown on a graph axis 440. The x acceleration values are shown on another axis 450 with the y acceleration values 452 and z acceleration values 454 shown as well. In some embodiments, accelerometer data for multiple individuals may be displayed when desired or shown on an aggregated basis. Markers and annotations may be included and used similarly to those discussed in FIG. 3.

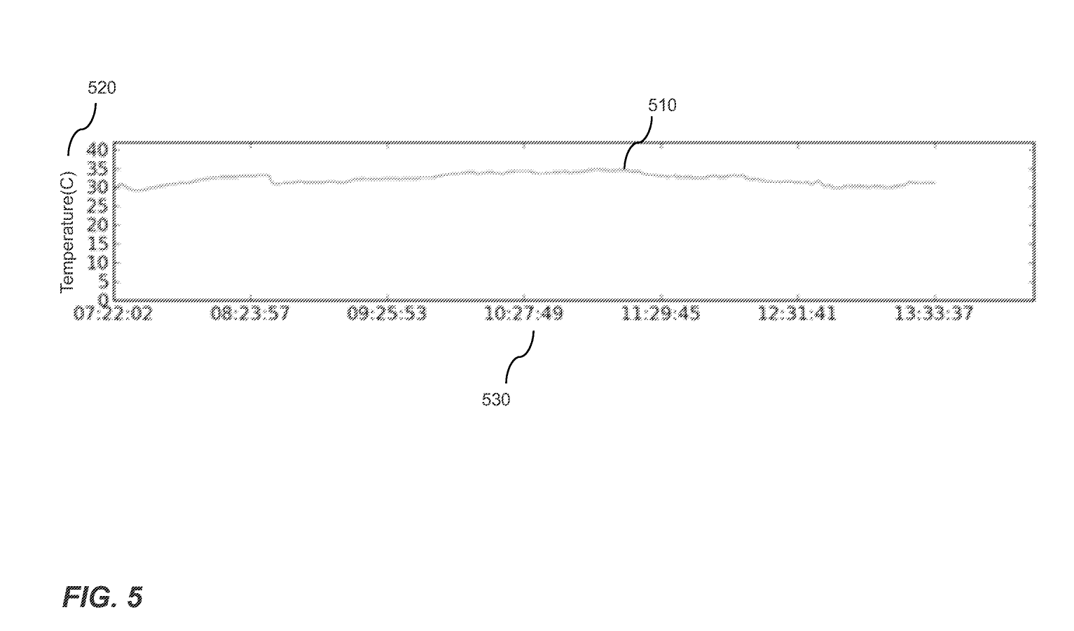

FIG. 5 is a graphical rendering of skin temperature data. A graph line 510 shows the electrodermal activity collected for an individual. The value for skin temperature is shown on the y-axis 520 for the graph. The skin temperature value was collected over a period of time and the timescale 530 is shown on the x-axis of the graph. In some embodiments, skin temperature values for multiple individuals may be displayed when desired or shown on an aggregated basis. Markers and annotations may be included and used similarly to those discussed in FIG. 3.

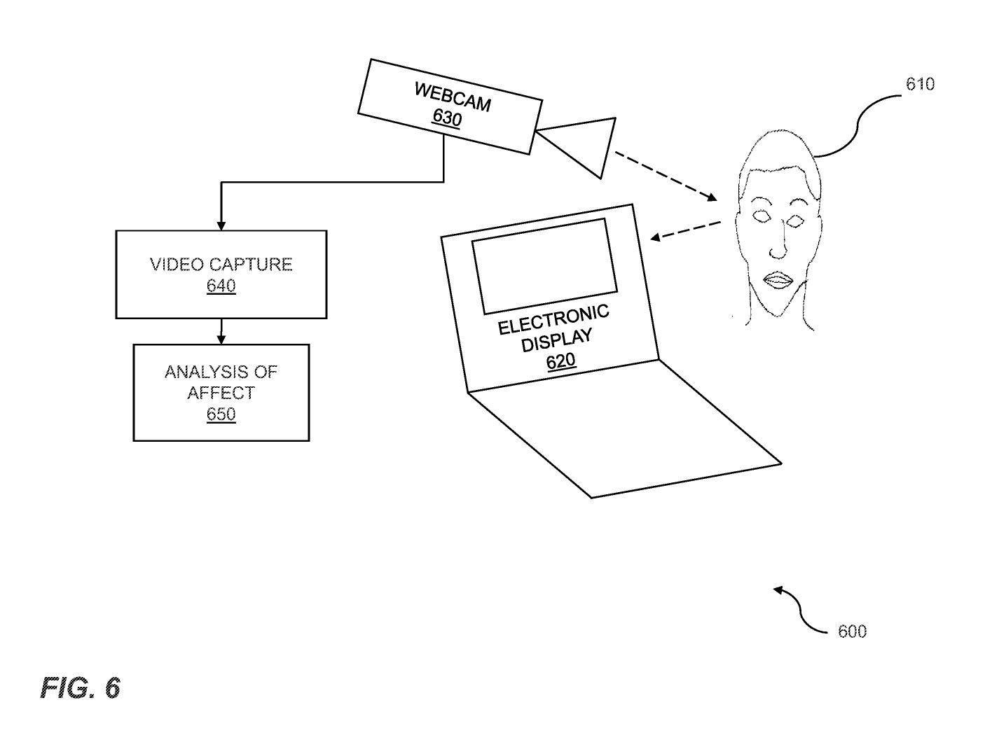

FIG. 6 shows an image collection system for facial analysis. A system 600 includes an electronic display 620 and a webcam 630. The system 600 captures facial response to the electronic display 620. In some embodiments, the system 600 captures facial responses to other stimuli such as a store display, an automobile ride, a board game, movie screen, or other type experience. The facial data may include video and collection of information relating to mental states. In some embodiments, a webcam 630 may capture video of the person 610. The video may be captured 530 onto a disk, tape, into a computer system, or streamed to a server. Images or a sequence of images of the person 610 may be captured by video camera, web camera still shots, thermal imager, CCD devices, phone camera, or other camera type apparatus.

The electronic display 620 may show a video or other presentation. The electronic display 620 may include a computer display, a laptop screen, a mobile device display, a cell phone display, or some other electronic display. The electronic display 620 may include a keyboard, mouse, joystick, touchpad, touch screen, wand, motion sensor, and other input means. The electronic display 620 may show a webpage, a website, a web-enabled application, or the like. The images of the person 610 may be captured by a video capture unit 640. In some embodiments, video of the person 610 is captured while in others a series of still images are captured. In embodiments, a webcam is used to capture the facial data.

Analysis of action units, gestures, and mental states may be accomplished using the captured images of the person 610. The action units may be used to identify smiles, frowns, and other facial indicators of mental states. In some embodiments, smiles are directly identified and in some cases the degree of smile (small, medium, and large for example) may be identified. The gestures, including head gestures, may indicate interest or curiosity. For example, a head gesture of moving toward the electronic display 620 may indicate increased interest or a desire for clarification. Facial analysis 650 may be performed based on the information and images which are captured. The analysis can include facial analysis and analysis of head gestures. Based on the captured images, analysis of physiology may be performed. The evaluating of physiology may include evaluating heart rate, heart rate variability, respiration, perspiration, temperature, skin pore size, and other physiological characteristics by analyzing images of a person's face or body. In many cases the evaluating may be accomplished using a webcam. Additionally, in some embodiments, physiology sensors may be attached to the person to obtain further data on mental states.

The analysis may be performed in real time or just in time. In some embodiments analysis is scheduled and then run through an analyzer or a computer processor which has been programmed to perform facial analysis. In some embodiments the computer processor may be aided by human intervention. The human intervention may identify mental states which the computer processor did not. In some embodiments the processor identifies places where human intervention is useful while in other embodiments the human reviews the facial video and provides input even when the processor did not identify that intervention was useful. In some embodiments the processor may perform machine learning based on the human intervention. Based on the human input the processor may learn that certain facial action units or gestures correspond to specific mental states and then be able to identify these mental states in an automated fashion without human intervention in the future.

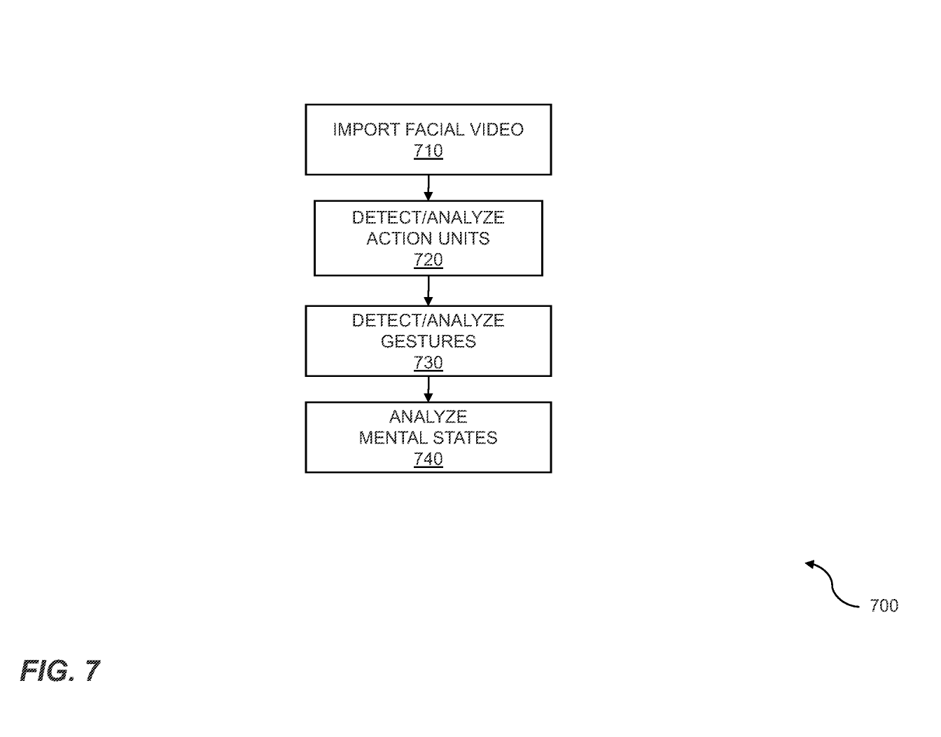

FIG. 7 is a flowchart for performing facial analysis. Flow 700 may begin with importing of facial video 710. The facial video may have been previously recorded and stored for later analysis. Alternatively, the importing of facial video may occur real time as an individual is being observed. Action units may be detected and analyzed 720. Action units may include the raising of an inner eyebrow, tightening of the lip, lowering of the brow, flaring of nostrils, squinting of the eyes, and many other possibilities. These action units may be automatically detected by a computer system analyzing the video. Alternatively, small regions of motion of the face that are not traditionally numbered on formal lists of action units may also be considered as action units for input to the analysis, such as a twitch of a smile or an upward movement above both eyes. Alternatively a combination of automatic detection by a computer system and human input may be provided to enhance the detection of the action units or related input measures. Facial and head gestures may be detected and analyzed 730. Gestures may include tilting the head to the side, leaning forward, a smile, a frown, as well as many other gestures. An analysis of mental states 740 may be performed. The mental states may include happiness, sadness, concentration, confusion, as well as many others. Based on the action units and facial or head gestures mental states may be analyzed, inferred, and identified.

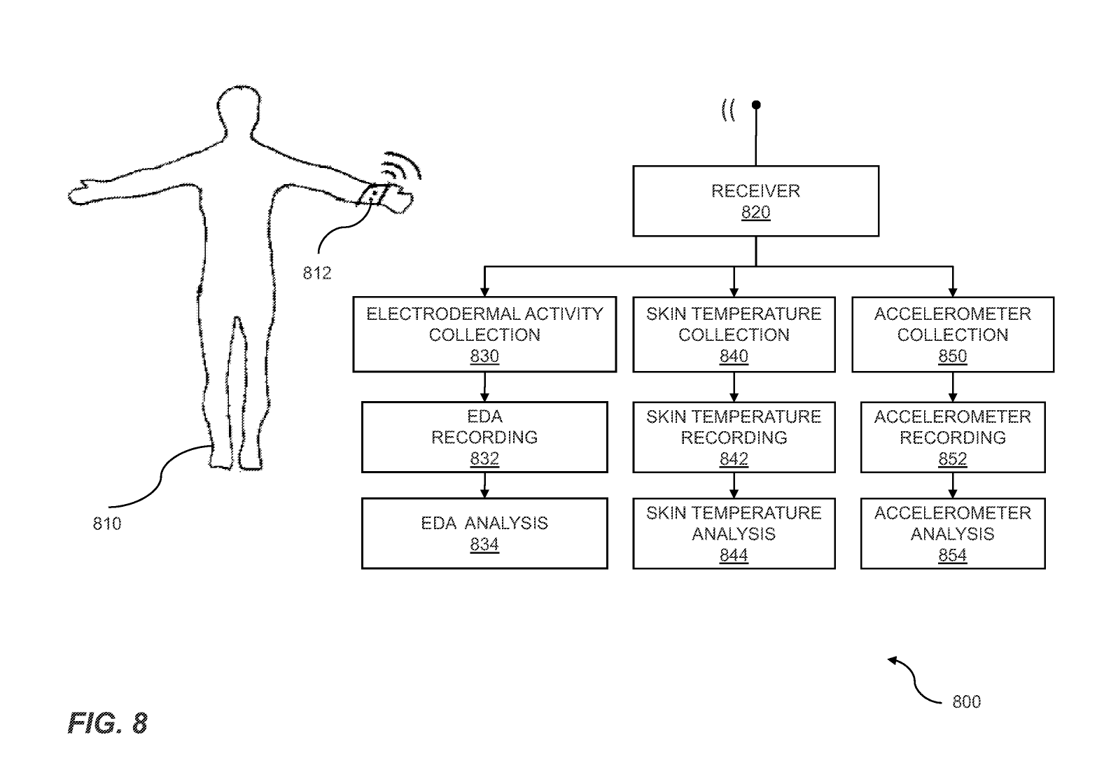

FIG. 8 is a diagram describing physiological analysis. A system 800 may analyze a person 810 for whom data is being collected. The person 810 may have a sensor 812 attached to him or her. The sensor 812 may be placed on the wrist, palm, hand, head, sternum, or other part of the body. In some embodiments, multiple sensors are placed on a person, such as for example on both wrists. The sensor 812 may include detectors for electrodermal activity, skin temperature, and accelerometer readings. Other detectors may be included as well such as heart rate, blood pressure, and other physiological detectors. The sensor 812 may transmit information collected to a receiver 820 using wireless technology such as Wi-Fi, Bluetooth, 802.11, cellular, or other bands. In some embodiments, the sensor 812 may store information and burst download the data through wireless technology. In other embodiments, the sensor 812 may store information for later wired download. The receiver may provide the data to one or more components in the system 800. Electrodermal activity (EDA) may be collected 830. Electrodermal activity may be collected continuously, every second, four times per second, eight times per second, 32 times per second, or on some other periodic basis or based on some event. The electrodermal activity may be recorded 832. The recording may be to a disk, a tape, onto a flash drive, into a computer system, or streamed to a server. The electrodermal activity may be analyzed 834. The electrodermal activity may indicate arousal, excitement, boredom, or other mental states based on changes in skin conductance.

Skin temperature may be collected 840 continuously, every second, four times per second, eight times per second, 32 times per second, or on some other periodic basis. The skin temperature may be recorded 842. The recording may be to a disk, a tape, onto a flash drive, into a computer system, or streamed to a server. The skin temperature may be analyzed 844. The skin temperature may used to indicate arousal, excitement, boredom, or other mental states based on changes in skin temperature.

Accelerometer data may be collected 850. The accelerometer may indicate one, two, or three dimensions of motion. The accelerometer data may be recorded 852. The recording may be to a disk, a tape, onto a flash drive, into a computer system, or streamed to a server. The accelerometer data may be analyzed 854. The accelerometer data may be used to indicate a sleep pattern, a state of high activity, a state of lethargy, or other state based on accelerometer data.

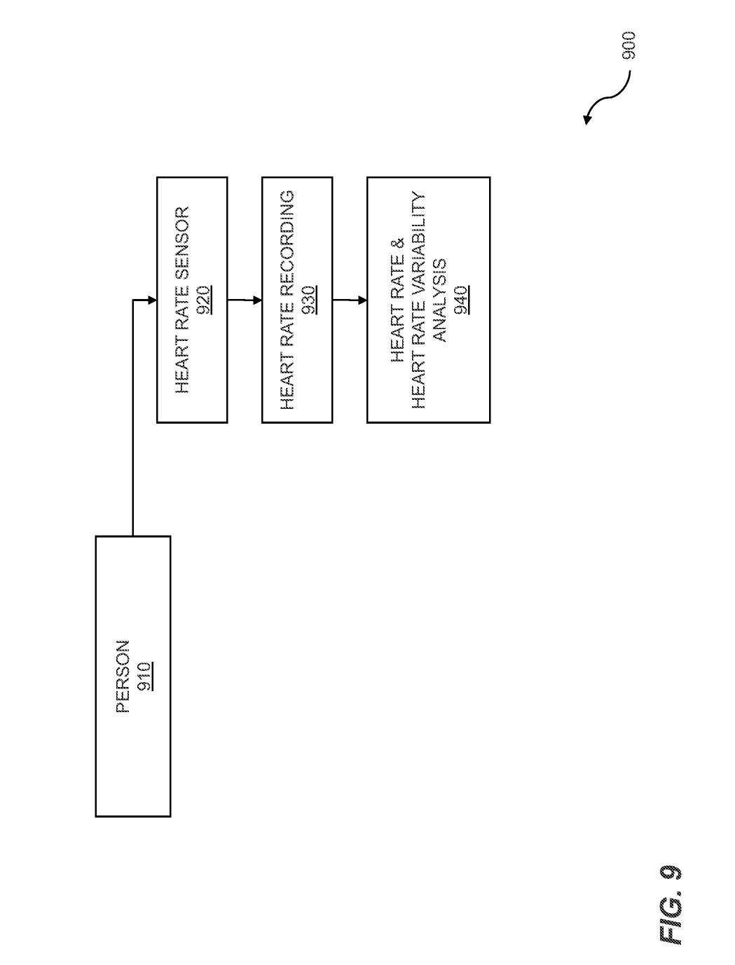

FIG. 9 is a diagram describing heart rate analysis. A person 910 may be observed. The person may be observed by a heart rate sensor 920. The observation may be through a contact sensor, through video analysis which enables capture of heart rate information, or other contactless sensing. The heart rate may be recorded 930. The recording may be to a disk, a tape, onto a flash drive, into a computer system, or streamed to a server. The heart rate and heart rate variability may be analyzed 940. An elevated heart rate may indicate excitement, nervousness, or other mental states. A lowered heart rate may be used to indicate calmness, boredom, or other mental states. A heart rate being variable may indicate good health and lack of stress. A lack of heart rate variability may indicate an elevated level of stress.

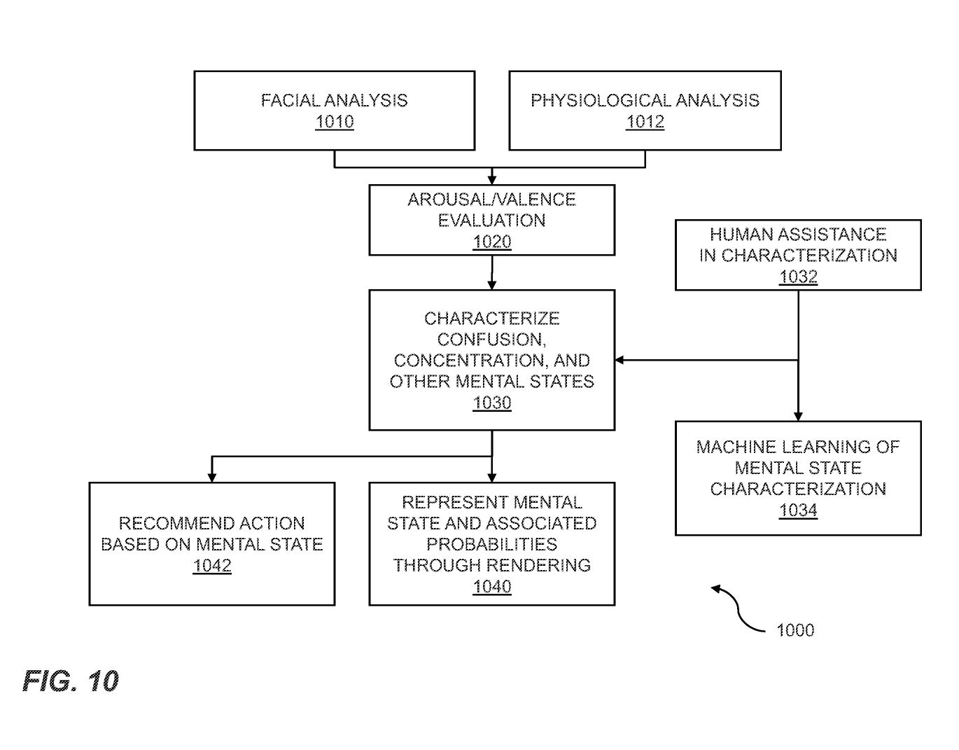

FIG. 10 is a flowchart for performing mental state analysis and rendering. The flow 1000 may begin with various types of data collection and analysis. Facial analysis 1010 may be performed, identifying action units, facial and head gestures, smiles, and mental states. Physiological analysis 1012 may be performed. The physiological analysis may include electrodermal activity, skin temperature, accelerometer data, heart rate, and other measurements related to the human body. The physiological data may be collected through contact sensors, through video analysis as in the case of heart rate information, or other means. In some embodiments, an arousal and valence evaluation 1020 may be performed. A level of arousal may range from calm to excited. A valence may be a positive or a negative predisposition. The combination of valence and arousal may be used to characterize mental states 1030. The mental states may include confusion, concentration, happiness, contentedness, confidence, as well as other states.

In some embodiments the characterization of mental states 1030 may be completely evaluated by a computer system. In other embodiments human assistance may be provided in inferring the mental state 1032. The process may involve using a human to evaluate a portion of one of a group comprising facial expressions, head gestures, hand gestures, and body language. A human may be used to evaluate only a small portion or even a single expression or gesture. Thus a human may evaluate a small portion of the facial expressions, head gestures, or hand gestures. Likewise a human may evaluate a portion of the body language of the person being observed. In embodiments, the process may involve prompting a human for input on an evaluation of the mental state for a section of the data which was captured. A human may view the facial analysis or physiological analysis raw data including video or may view portions of the raw data or analyzed results. The human may intervene and provide input to aid in inferring of the mental state or may identify the mental state to the computer system used in the characterization of the mental state 1030. A computer system may highlight the portions of data where human intervention is needed and may jump to the point in time where the data for that needed intervention may be presented to the human. A feedback may be provided to a human that provides assistance in characterization. Multiple people may provide assistance in characterizing mental states. Based on the automated characterization of mental states as well as evaluation by multiple humans, feedback may be provided to a human to improve the human's accuracy in characterization. Individual humans may be compensated for providing assistance in characterization. Improved accuracy in characterization, based on the automated characterization or based on the other people assisting in characterization, may result in enhanced compensation.

The flow 1000 may include learning by the computer system. Machine learning of the mental state evaluation 1034 may be performed by the computer system used in the characterization of the mental state 1030. The machine learning may be based on the input from the human on the evaluation of the mental state for the section of data.

A representation of the mental state and associated probabilities may be rendered 1040. The mental state may be presented on a computer display, electronic display, cell phone display, personal digital assistance screen, or other display. The mental state may be displayed graphically. A series of mental states may be presented with the likelihood of each state for a given point in time. Likewise a series of probabilities for each mental state may be presented over the timeline for which facial and physiological data was analyzed. In some embodiments an action may be recommended based on the mental state 1042 which was detected. An action may include recommending a question in a focus group session. An action may be changing an advertisement on a web page. An action may be editing a movie which was viewed to remove an objectionable section or boring portion. An action may be moving a display in a store. An action may be editing a confusing section of a tutorial on the web or in a video.

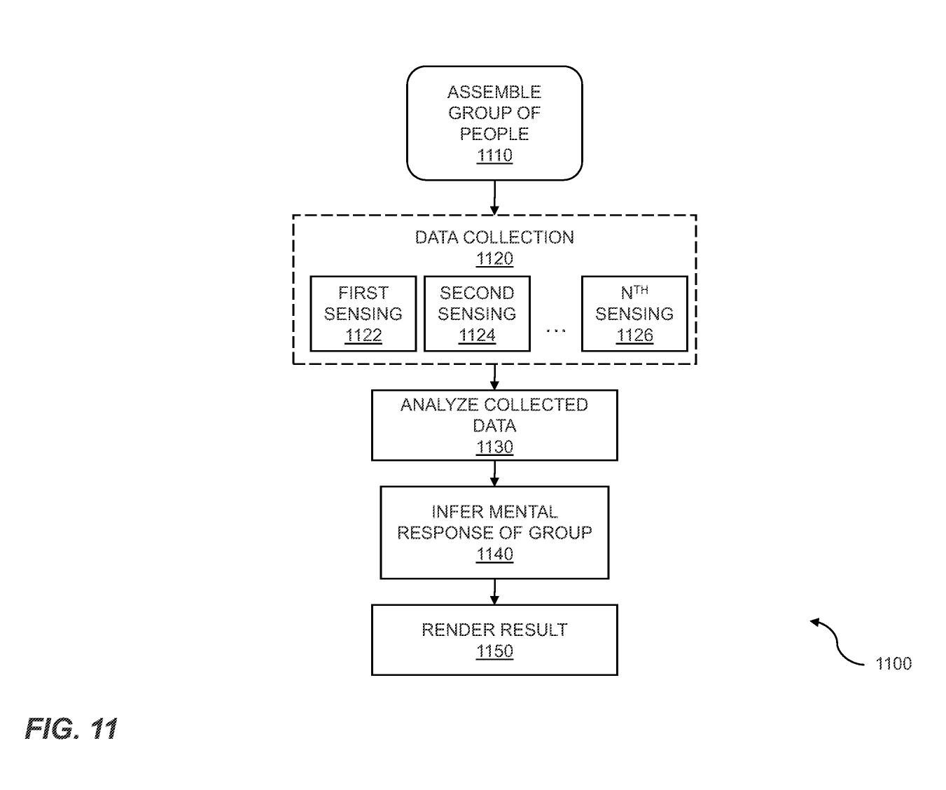

FIG. 11 is a flowchart describing analysis of the mental response of a group. The flow 1100 may begin with assembling a group of people 1110. The group of people may have a common experience such as viewing a movie, viewing a television show, viewing a movie trailer, viewing a streaming video, viewing an advertisement, listening to a song, viewing or listening to a lecture, using a computer program, using a product, consuming a food, using a video or computer game, education through a distance learning, riding in or driving a transportation vehicle such as a car, or some other experience. Data collection 1120 may be performed on each member of the group of people 1110. A plurality of sensings may occur on each member of the group of people 1110 including, for example, a first sensing 1122, a second sensing 1124, and so on through an n^{th} sensing 1126. The various sensings for which data collection 1120 is performed may include capturing facial expressions, electrodermal activity, skin temperature, accelerometer readings, heart rate, as well as other physiological information. The data which was captured may be analyzed 1130. This analysis may include characterization of arousal and valence as well as characterization of mental states for each of the individuals in the group of people 1110. The mental response of the group may be inferred 1140 providing a collective mental state. The mental states may be summarized to evaluate the common experience of all of the individuals in the group of people 1110. A result may be rendered 1150. The result may be a function of time or a function of the sequence of events experienced by the people. The result may include a graphical display of the valence and arousal. The result may include a graphical display of the mental states of the individuals and the group collectively.

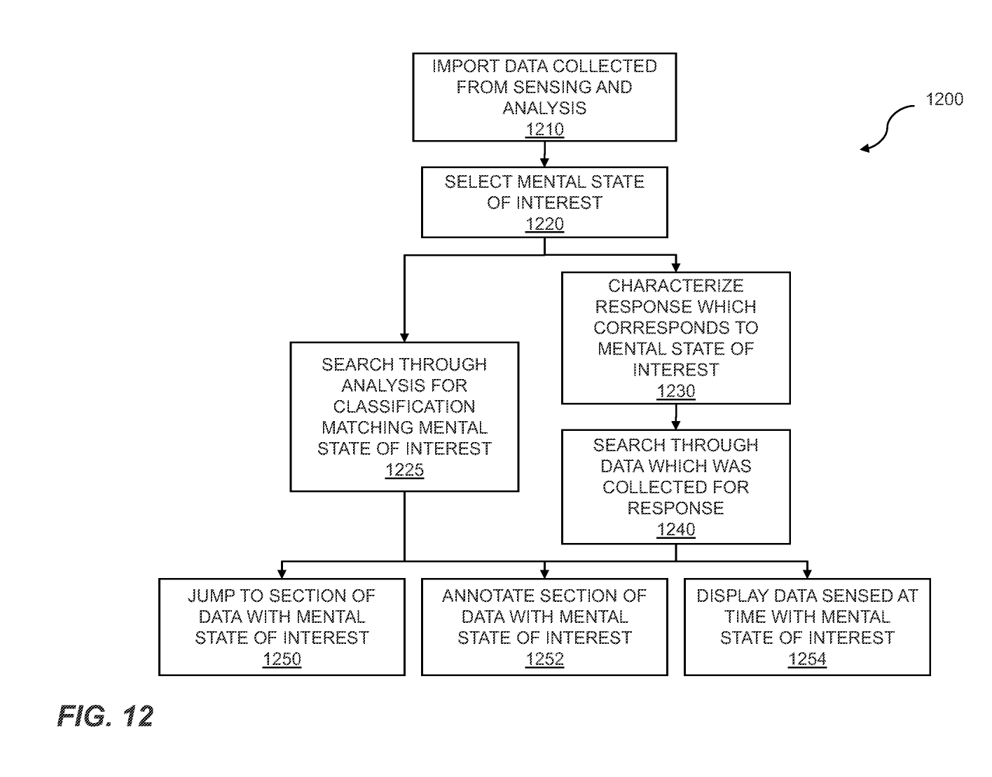

FIG. 12 is a flowchart for identifying data portions which match a selected mental state of interest. The flow 1200 may begin with an import of data collected from sensing along with any analysis performed to date 1210. The importing of data may be the loading of stored data which was previously captured or may be the loading of data which is captured real time. The data may also already exist within the system doing the analysis. The sensing may include capture of facial expressions, electrodermal activity, skin temperature, accelerometer readings, heart rate capture, as well as other physiological information. Analysis may be performed on the various data collected from sensing to characterizing mental states.

A mental state that interests the user may be selected 1220. The mental state of interest may be confusion, concentration, confidence, delight as well as many others. In some embodiments, analysis may have been previously performed on the data which was collected. The analysis may include indexing of the data and classifying mental states which were inferred or detected. When analysis has been previously performed and the mental state of interest has already been classified, a search through the analysis for one or more classifications matching the selected state may be performed 1225. By way of example, confusion may have been selected as the mental state of interest. The data which was collected may have been previously analyzed for various mental states, including confusion. When the data which was collected was indexed, a classification for confusion may have been tagged at various points in time during the data collection. The analysis may then be searched for any confusion points as they have already been classified previously.

In some embodiments, a response may be characterized which corresponds to the mental state of interest 1230. The response may be a positive valence and being aroused, as in an example where confidence is selected as the mental state of interest. The response may be reduced to valence and arousal or may be reduced further to look for action units or facial expressions and head gestures.

The data which was collected may be searched through for a response 1240 corresponding to the selected state. The sensed data may be searched or analysis derived from the collected data may be searched. The search may look for action units, facial expressions, head gestures, or mental states which match the selected state for which the user is interested 1220.

The section of data with the mental state of interest may be jumped to 1250. For example, when confusion is selected, the data or analysis derived from the data may be shown corresponding to the point in time where confusion was exhibited. This “jump to feature” may be thought of as a fast forward through the data to the interesting section where confusion or another selected mental state is detected. When facial video is considered, the key sections of the video which match the selected state may be displayed. In some embodiments, the section of the data with the mental state of interest may be annotated 1252. Annotations may be placed along the timeline marking the data and the times with the selected state. In embodiments, the data sensed at the time with the selected state may be displayed 1254. The data may include facial video. The data may also include graphical representation of electrodermal activity, skin temperature, accelerometer readouts, heart rate, and other physiological readings.

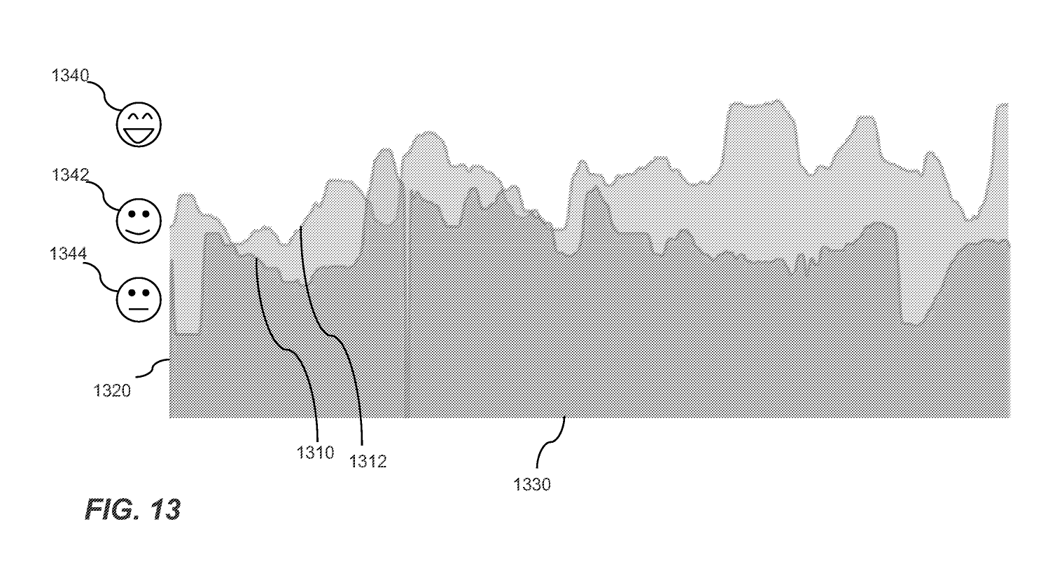

FIG. 13 is a graphical rendering of mental state analysis along with an aggregated result from a group of people. This rendering may be displayed on a web page, web enabled application, or other type of electronic display representation. A graph 1310 may be shown for an individual on whom affect data is collected. The mental state analysis may be based on facial image or physiological data collection. In some embodiments, the graph 1310 may indicate the amount or probability of a smile being observed for the individual. A higher value or point on the graph may indicate a stronger or larger smile. In certain spots the graph may drop out or degrade when image collection lost or was not able to identify the face of the person. The probability or intensity of an affect may be given along the y-axis 1320. A timeline may be given along the x-axis 1330. Another graph 1312 may be shown for affect collected on another individual or aggregated affect from multiple people. The aggregated information may be based on taking the average, median, or other collected value from a group of people. In some embodiments, graphical smiley face icons 1340, 1342, and 1344 may be shown providing an indication of the amount of a smile or other facial expression. A first very broad smiley face icon 1340 may indicate a very large smile being observed. A second normal smiley face icon 1342 may indicate a smile being observed. A third face icon 1340 may indicate no smile. Each of the icons may correspond to a region on the y-axis 1320 that indicate the probability or intensity of a smile.

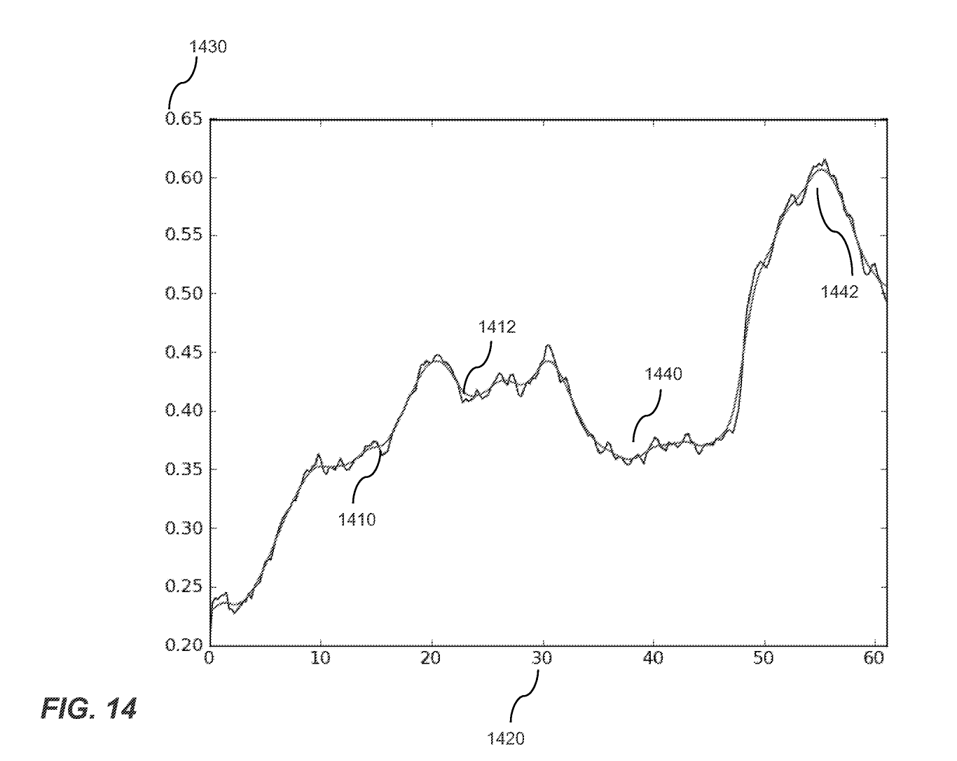

FIG. 14 is a graphical rendering of mental state analysis. This rendering may be displayed on a web page, web enabled application, or other type of electronic display representation. A graph 1410 may indicate the observed affect intensity or probability of occurring. A timeline may be given along the x-axis 1420. The probability or intensity of an affect may be given along the y-axis 1430. A second graph 1412 may show a smoothed version of the graph 1410. One or more valleys in the affect may be identified such as the valley 1440. One or more peaks in affect may be identified such as the peak 1442.

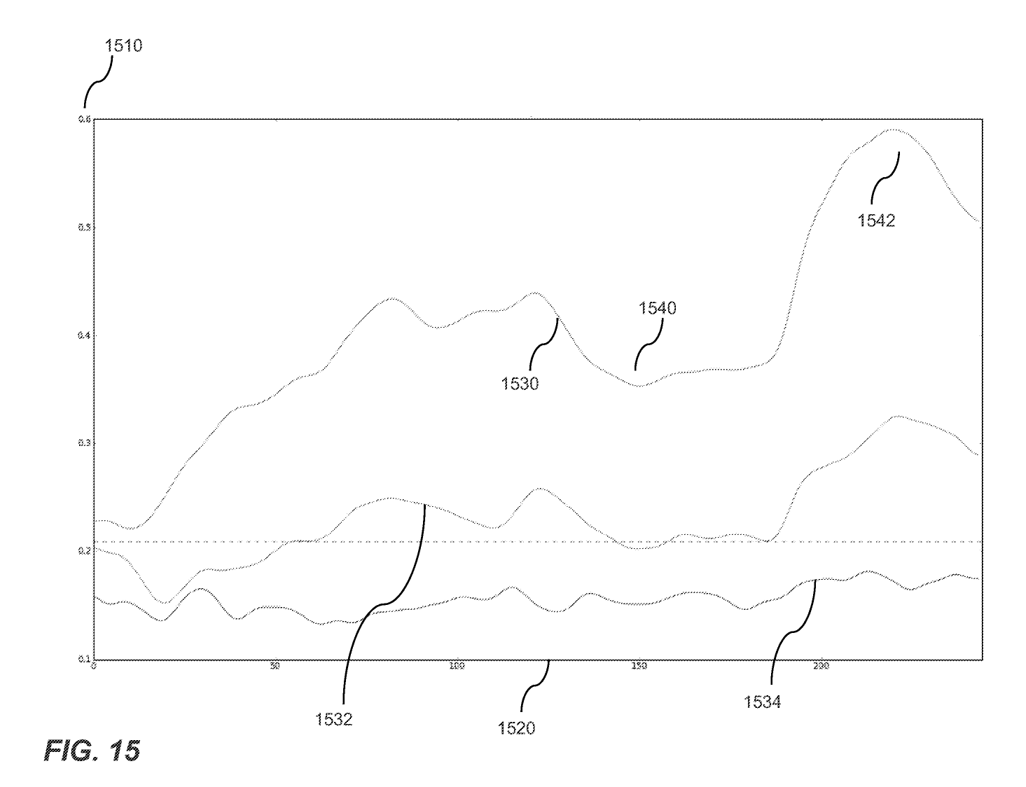

FIG. 15 is a graphical rendering of mental state analysis based on metadata. This rendering may be displayed on a web page, web enabled application, or other type of electronic display representation. On a graph a first line 1530, a second line 1532, and a third line 1534 may each correspond to different metadata collected. For instance, self-reporting metadata may be collected for whether the person reported that they “really liked”, “liked”, or “was ambivalent” about a certain event. The event could be a movie, a television show, a web series, a webisode, a video, a video clip, an electronic game, an advertisement, an e-book, an e-magazine, or the like. The first line 1530 may correspond to an event a person “really liked” while the second line 1532 may correspond to another person who “liked the event. Likewise, the third line 1534 may correspond to a different person who “was ambivalent” to the event. In some embodiments, the lines could correspond to aggregated results of multiple people.

Each of the above methods may be executed on one or more processors on one or more computer systems. Embodiments may include various forms of distributed computing, client/server computing, and cloud based computing. Further, it will be understood that for each flow chart in this disclosure, the depicted steps or boxes are provided for purposes of illustration and explanation only. The steps may be modified, omitted, or re-ordered and other steps may be added without departing from the scope of this disclosure. Further, each step may contain one or more sub-steps. While the foregoing drawings and description set forth functional aspects of the disclosed systems, no particular arrangement of software and/or hardware for implementing these functional aspects should be inferred from these descriptions unless explicitly stated or otherwise clear from the context. All such arrangements of software and/or hardware are intended to fall within the scope of this disclosure.

The block diagrams and flowchart illustrations depict methods, apparatus, systems, and computer program products. Each element of the block diagrams and flowchart illustrations, as well as each respective combination of elements in the block diagrams and flowchart illustrations, illustrates a function, step or group of steps of the methods, apparatus, systems, computer program products and/or computer-implemented methods. Any and all such functions may be implemented by computer program instructions, by special-purpose hardware-based computer systems, by combinations of special purpose hardware and computer instructions, by combinations of general purpose hardware and computer instructions, by a computer system, and so on. Any and all of which may be generally referred to herein as a “circuit,” “module,” or “system.”

A programmable apparatus that executes any of the above mentioned computer program products or computer implemented methods may include one or more processors, microprocessors, microcontrollers, embedded microcontrollers, programmable digital signal processors, programmable devices, programmable gate arrays, programmable array logic, memory devices, application specific integrated circuits, or the like. Each may be suitably employed or configured to process computer program instructions, execute computer logic, store computer data, and so on.

It will be understood that a computer may include a computer program product from a computer-readable storage medium and that this medium may be internal or external, removable and replaceable, or fixed. In addition, a computer may include a Basic Input/Output System (BIOS), firmware, an operating system, a database, or the like that may include, interface with, or support the software and hardware described herein.

Embodiments of the present invention are not limited to applications involving conventional computer programs or programmable apparatus that run them. It is contemplated, for example, that embodiments of the presently claimed invention could include an optical computer, quantum computer, analog computer, or the like. A computer program may be loaded onto a computer to produce a particular machine that may perform any and all of the depicted functions. This particular machine provides a means for carrying out any and all of the depicted functions.

Any combination of one or more computer readable media may be utilized. The computer readable medium may be a non-transitory computer readable medium for storage. A computer readable storage medium may be electronic, magnetic, optical, electromagnetic, infrared, semiconductor, or any suitable combination of the foregoing. Further computer readable storage medium examples may include an electrical connection having one or more wires, a portable computer diskette, a hard disk, a random access memory (RAM), a read-only memory (ROM), an erasable programmable read-only memory (EPROM), Flash, MRAM, FeRAM, phase change memory, an optical fiber, a portable compact disc read-only memory (CD-ROM), an optical storage device, a magnetic storage device, or any suitable combination of the foregoing. In the context of this document, a computer readable storage medium may be any tangible medium that can contain or store a program for use by or in connection with an instruction execution system, apparatus, or device.

It will be appreciated that computer program instructions may include computer executable code. A variety of languages for expressing computer program instructions may include without limitation C, C++, Java, JavaScript™, ActionScript™, assembly language, Lisp, Perl, Tcl, Python, Ruby, hardware description languages, database programming languages, functional programming languages, imperative programming languages, and so on. In embodiments, computer program instructions may be stored, compiled, or interpreted to run on a computer, a programmable data processing apparatus, a heterogeneous combination of processors or processor architectures, and so on. Without limitation, embodiments of the present invention may take the form of web-based computer software, which includes client/server software, software-as-a-service, peer-to-peer software, or the like.

In embodiments, a computer may enable execution of computer program instructions including multiple programs or threads. The multiple programs or threads may be processed more or less simultaneously to enhance utilization of the processor and to facilitate substantially simultaneous functions. By way of implementation, any and all methods, program codes, program instructions, and the like described herein may be implemented in one or more thread. Each thread may spawn other threads, which may themselves have priorities associated with them. In some embodiments, a computer may process these threads based on priority or other order.

Unless explicitly stated or otherwise clear from the context, the verbs “execute” and “process” may be used interchangeably to indicate execute, process, interpret, compile, assemble, link, load, or a combination of the foregoing. Therefore, embodiments that execute or process computer program instructions, computer-executable code, or the like may act upon the instructions or code in any and all of the ways described. Further, the method steps shown are intended to include any suitable method of causing one or more parties or entities to perform the steps. The parties performing a step, or portion of a step, need not be located within a particular geographic location or country boundary. For instance, if an entity located within the United States causes a method step, or portion thereof, to be performed outside of the United States then the method is considered to be performed in the United States by virtue of the entity causing the step to be performed.

While the invention has been disclosed in connection with preferred embodiments shown and described in detail, various modifications and improvements thereon will become apparent to those skilled in the art. Accordingly, the spirit and scope of the present invention is not to be limited by the foregoing examples, but is to be understood in the broadest sense allowable by law.

What is claimed is:

1. A computer implemented method for analyzing mental states comprising:
capturing data on an individual into a computer system wherein the data provides information for evaluating a mental state of the individual;
receiving analysis from a web service wherein the analysis is based on the data on the individual which was captured; and
rendering an output which describes the mental state of the individual based on the analysis which was received.

2. The method of claim 1 wherein the data on the individual includes one of a group comprising facial expressions, physiological information, and accelerometer readings.

3. The method of claim 2 wherein the facial expressions further comprise head gestures.

4. The method of claim 2 wherein the physiological information includes one of a group comprising electrodermal activity, heart rate, heart rate variability, and respiration.

5. The method of claim 2 wherein the physiological information is collected without contacting the individual.

6. The method of claim 1 wherein the mental state is one of a cognitive state and an emotional state.

7. The method of claim 1 wherein the web service comprises an interface which includes a server that is remote to the individual and cloud-based storage.

8. The method of claim 1 further comprising indexing the data on the individual through the web service.

9. The method of claim 8 wherein the indexing includes categorization based on valence and arousal information.

10. The method of claim 1 further comprising receiving analysis information on a plurality of other people wherein the analysis information allows evaluation of a collective mental state of the plurality of other people.

11. The method of claim 10 wherein the analysis information includes correlation for the mental state of the plurality of other people to the data which was captured on the mental state of the individual.

12. The method of claim 11 wherein the correlation is based on metadata from the individual and metadata from the plurality of other people.

13. The method of claim 1 wherein the analysis which is received from the web service is based on specific access rights.

14. The method of claim 1 further comprising sending a request to the web service for the analysis.

15. The method of claim 14 wherein the analysis is generated just in time based on a request for the analysis.

16. The method of claim 1 further comprising sending a subset of the data which was captured on the individual to the web service.

17. The method of claim 1 wherein the rendering is based on data which is received from the web service.

18. The method of claim 17 wherein the data which is received includes a serialized object in a form of JavaScript Object Notation (JSON).

19. The method of claim 18 further comprising deserializing the serialized object into a form for a JavaScript object.

20. The method of claim 1 wherein the rendering further comprises recommending a course of action based on the mental state of the individual.

21. The method of claim 20 wherein the recommending includes one of a group comprising modifying a question queried to a focus group, changing an advertisement on a web page, editing a movie which was viewed to remove an objectionable section, changing direction of an electronic game, changing a medical consultation presentation, and editing a confusing section of an internet-based tutorial.

22. A computer program product embodied in a non-transitory computer readable medium for analyzing mental states, the computer program product comprising:
code for capturing data on an individual into a computer system wherein the data provides information for evaluating a mental state of the individual;
code for receiving analysis from a web service wherein the analysis is based on the data on the individual which was captured; and
code for rendering an output which describes the mental state of the individual based on the analysis which was received.

23. A system for analyzing mental states comprising:
a memory which stores instructions;
one or more processors attached to the memory wherein the one or more processors, when executing the instructions which are stored, are configured to:
capture data on an individual wherein the data provides information for evaluating a mental state of the individual;
receive analysis from a web service wherein the analysis is based on the data on the individual which was captured; and
render an output which describes the mental state of the individual based on the analysis which was received.
